(19) United States
(12) Patent Application Publication
(10) Pub. No.: US 2005/0119337 A1
(43) Pub. Date: Jun. 2, 2005

(54) Methods of application of Schisandrin B in the preparation of anticancer medications

(76) Inventor: Xun Hu, Hangzhou (CN)

Correspondence Address: CHAN LAW GROUP LC, 1055 W. 7TH ST,, SUITE 1880, LOS ANGELES, CA, 90017, US

(21) Appl. No.: 10/998,025

(22) Filed: Nov. 24, 2004

(30) Foreign Application Priority Data
Nov. 28, 2003 (CN) 200310108996X
Jun. 11, 2004 (CN) 2004100596073

(51) Int. Cl. A61K 38/16; A61K 31/7048; A61K 31/704; A61K 31/7072; A61K 31/522; A61K 31/335; A61K 31/34

(52) U.S. Cl. 514/462; 514/8; 514/109; 514/49; 514/34; 514/27; 514/283; 514/492; 424/649; 514/171; 514/263.31

(57) ABSTRACT

Methods of application of Schisandrin B in the preparation of anticancer medications, and particularly for the preparation of medications for the treatment of multidrug resistant (MDR) cancer. The compound of Schisandrin B effectively reverses MDR cancer in combination with other anticancer chemotherapeutic agents. Schisandrin B reverses MDR cancer by inhibiting the drug efflux activity of P-glycoprotein, indicating its significance in clinical applications. Although it is of low toxicity, Schisandrin B is cytotoxic to human cancer cells, revealing its application in cancer chemotherapy. It is emphasized that this abstract is provided to comply with the rules requiring an abstract that will allow a searcher or other reader to quickly ascertain the subject matter of the technical disclosure. It is submitted with the understanding that it will not be used to interpret or limit the scope or meaning of the claims.

### RELATED APPLICATIONS

This application claims the priority of Application Nos. 200310108996X and 2004100596073, filed in China on Nov. 28, 2003 and Jun. 11, 2004, respectively, pursuant to 35 U.S.C. § 119.

### BACKGROUND OF THE INVENTION

1. Field of the Invention

This disclosure relates to a novel application of the chemical compound Schisandrin B, and more specifically to the application of Schisandrin B in the preparation of medications as a cancer chemotherapeutic agent and as a cancer multidrug resistant (MDR) reversal agent.

2. Description of the Prior Art

Cancer is the leading cause of death. Chemotherapy is one of the primary ways to treat cancer. However, a major problem with chemotherapy is the ability of cancer cells to develop resistance to the cytotoxic effects of anticancer drugs during treatment. Previous studies have shown that cancer cells have the ability to become simultaneously resistant to several chemotherapeutic drugs having unrelated chemical structures and mechanisms of action. This phenomenon is commonly referred to as multidrug resistance (MDR). It has been reported that a clinically relevant and scientifically documented mechanism for MDR in cancer cells is associated with the expression of P-glycoprotein.

P-glycoprotein, having a molecular weight of 170 kD, is a transmembrane protein that universally transports intracellular drugs out of the cell by catalyzing the hydrolysis of ATP. Current research tends to show that the overexpression of P-glycoprotein causes the rapid efflux of intracellular drugs, resulting in a decreased accumulation of anticancer drugs within MDR cancer cells. One of the ways to overcome MDR cancer is to inhibit the drug-pump function of P-glycoprotein by use of chemical inhibitors. As MDR reversal agents are presently lacking in clinical cancer therapy, there is a strong need to develop highly effective P-glycoprotein inhibitors to clinically applicable drugs.

One of the earlier identified potent P-glycoprotein inhibitors is verapamil. However, verapamil causes severe side effects such as cardiovascular toxicity that hinder its clinical application.

It is therefore desirable to provide a compound that is useful for treating MDR cancer cells, particularly as a potent P-glycoprotein inhibitor for clinically applicable drugs.

### SUMMARY OF THE INVENTION

A method of treating cancer according to the present disclosure concerns the discovery that the compound, Schisandrin B, a derivative of dibenzocyclo octadiene, may be useful in reversing MDR cancer by inhibiting the drug transport function of P-glycoprotein and acting as a chemotherapeutic agent against cancer. More specifically, the disclosure may be useful in treating or preventing P-glycoprotein-mediated MDR.

Schisandrin B, a compound extracted from the Chinese Schisandra chinensis (Turcz.) Baill and Schisandra sphenanthera Rehd. et Wils. plant, was previously reported to have antioxidant properties and the ability to protect against chemical-induced liver damage. The chemical structure of Schisandrin B, a derivative of dibenzocyclo octadiene, is as follows: 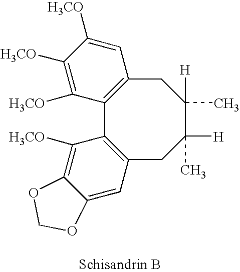
The role of Schisandrin B as an anticancer agent has not been previously reported.

Some advantages of the application of the compound, Schisandrin B, as an anticancer agent in medications is the compound's (1) potential in clinical applications as manifested by its potency in reversing MDR cancer by inhibiting P-glycoprotein; and (2) potential as a chemotherapeutic agent against cancer, as manifested by its low toxicity to normal human cells but relatively high toxicity to cancer cells.

This disclosure provides methods of application of the compound, Schisandrin B, which is of low toxicity but has strong potency in reversing MDR cancer, for use in the preparation of anticancer medications.

The embodiments described herein particularly demonstrate that Schisandrin B is of high potency in reversing MDR cancer and is comparable with verapamil. In comparison with verapamil, which is of high cardiovascular toxicity, Schisandrin B is of very low toxicity and very safe. Because of its desirable physiological properties, Schisandrin B has significant potential in clinical applications, such as the preparation of anticancer medications for the treatment of cancer.

In a first aspect, the present disclosure provides a method of application of the compound, Schisandrin B, in reversing MDR cancer that comprises preparing a medication, which comprises Schisandrin B. The preparation of the medication further comprises at least one anticancer chemotherapeutic agent and a pharmaceutically acceptable carrier. The chemotherapeutic agents are selected from the group consisting of doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, Schisandrin B, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof. The medication is formulated for administration in the form of a capsule, caplet, tablet, pill, suspension, or liquid. The medication includes at least one MDR reversal agent.

In another aspect, the present disclosure provides a method of application of the compound, Schisandrin B, for cancer therapy that comprises preparing a medication, which comprises Schisandrin B. Schisandrin B inhibits the drug transport function of P-glycoprotein in MDR cancer cells. Schisandrin B binds with P-glycoprotein by competing with anticancer agents. Schisandrin B enhances activities of the anticancer agents in inducing apoptosis of cancer cells.

In still another aspect, the present disclosure provides a method of application of the compound, Schisandrin B, for cancer chemotherapy that comprises preparing a medication, which comprises Schisandrin B.

These and other features and advantages of this disclosure will become further apparent from the detailed description and accompanying figures that follow.

### BRIEF DESCRIPTION OF THE DRAWINGS

FIG. 1 is a graph illustrating the reversal of resistance of K562/adr and K562/vcr to doxorubicin in the presence of Schisandrin B.

FIG. 2 is a graph illustrating the cytotoxicity of Schisandrin B toward seven types of cells.

FIG. 3 is a graph illustrating the accumulation of doxorubicin within multidrug resistant (MDR) cancer cell line K562/adr in the presence or absence of Schisandrin B or verapamil.

FIG. 4 is a graph illustrating the effects of Schisandrin B on the retention of doxorubicin in MDR cell K562/adr.

FIG. 5 is a graph illustrating the effects of Schisandrin B on daunorubicin accumulation in MDR cell K562/adr.

FIG. 6 is a graph illustrating Rh123 accumulation in MDR cell KBV200 in the absence and presence of Schisandrin B or verapamil.

FIG. 7A is a flow cytometric graph illustrating the expression of P-glycoprotein in drug sensitive cancer cell line K562 and MDR cancer cell line K562/adr.

FIG. 7B is a flow cytometric graph illustrating the expression of P-glycoprotein in drug sensitive cancer cell line K562 and MDR cancer cell line K562/vcr.

FIG. 7C is a flow cytometric graph illustrating the expression of P-glycoprotein in drug sensitive cancer cell line KB and MDR cancer cell line KBV200.

FIG. 8A is a photograph taken via fluorescent microscope illustrating the intracellular distribution of daunorubicin in KB cells.

FIG. 8B is a photograph taken via fluorescent microscope illustrating the intracellular distribution of daunorubicin in KBV200 cells.

FIG. 9A is a photograph taken via fluorescent microscope illustrating the intracellular distribution of Rh123 in KB cells.

FIG. 9B is a photograph taken via fluorescent microscope illustrating the intracellular distribution of Rh123 in KBV200 cells.

FIG. 10A is a photograph taken via fluorescent microscope at magnification ×100 illustrating the effects of verapamil (10 μg/ml) on the intracellular distribution of daunorubicin in KBV200 cells.

FIG. 10B is a photograph taken via fluorescent microscope at magnification ×200 illustrating the effects of verapamil (10 μg/ml) on the intracellular distribution of daunorubicin in KBV200 cells.

FIG. 10C is a photograph taken via fluorescent microscope at magnification ×100 illustrating the effects of Schisandrin B (5 μg/ml) on the intracellular distribution of daunorubicin in KBV200 cells.

FIG. 10D is a photograph taken via fluorescent microscope at magnification ×200 illustrating the effects of Schisandrin B (5 μg/ml) on the intracellular distribution of daunorubicin in KBV200 cells.

FIG. 10E is a photograph taken via fluorescent microscope at magnification ×100 illustrating the effects of Schisandrin B (10 μg/ml) on the intracellular distribution of daunorubicin in KBV200 cells.

FIG. 10F is a photograph taken via fluorescent microscope at magnification ×200 illustrating the effects of Schisandrin B (10 μg/ml) on the intracellular distribution of daunorubicin in KBV200 cells.

FIG. 11A is a photograph taken via fluorescent microscope at magnification ×400 illustrating the intracellular distribution of Rh123 in KBV200 cells in the absence of Schisandrin B or verapamil.

FIG. 11B is a photograph taken via fluorescent microscope at magnification ×400 illustrating the intracellular distribution of Rh123 in KBV200 cells in the presence of verapamil (10 μg/ml).

FIG. 11C is a photograph taken via fluorescent microscope at magnification ×400 illustrating the intracellular distribution of Rh123 in KBV200 cells in the presence of Schisandrin B (5 μg/ml).

FIG. 11D is a photograph taken via fluorescent microscope at magnification ×400 illustrating the intracellular distribution of Rh123 in KBV200 cells in the presence of Schisandrin B (10 μg/ml).

FIG. 12A is a photograph taken via fluorescent microscope illustrating untreated KBV200 cells.

FIG. 12B is a photograph taken via fluorescent microscope illustrating the apoptosis of KBV200 cells treated with doxorubicin (1 μg/ml).

FIG. 12C is a photograph taken via fluorescent microscope illustrating the apoptosis of KBV200 cells treated with doxorubicin

FIG. 12D is a photograph taken via fluorescent microscope illustrating the apoptosis of KBV200 cells treated with doxorubicin (1 μg/ml) and Schisandrin B (10 μg/ml).

FIG. 12E is a photograph taken via fluorescent microscope illustrating the apoptosis of KBV200 cells treated with doxorubicin (5 μg/ml) and Schisandrin B (10 μg/ml).

FIG. 12F is a photograph taken via fluorescent microscope illustrating KB cells treated with doxorubicin (1 μg/ml).

FIG. 13 is a photograph illustrating the effects of Schisandrin B on enhancing the activities of doxorubicin in the induction of apoptosis of KBV200.

FIG. 14 is a photograph illustrating the inhibition of ³H-azidopine binding with P-glycoprotein by Schisandrin B.

FIG. 15 is a graph illustrating the effects of combination treatment with Schisandrin B and doxorubicin on the growth of MDR cancer cell KBV200 in Balb/c nude mice.

FIG. 16 is a graph illustrating the effects of combination treatment with Schisandrin B and doxorubicin on the mean survival time of Balb/c nude mice bearing MDR cancer cell KBV200.

### DETAILED DESCRIPTION OF THE INVENTION

The terms and abbreviations used in the Detailed Description set forth herein have their normal meanings unless otherwise specified. For example, “° C.” refers to degrees Celsius; “mg” refers to milligram or milligrams; “ml” refers to milliliter or milliliters; “μg” refers to microgram or micrograms; “kg” refers to kilograms; and “IC₅₀” refers to the inhibitory concentration of a drug that causes 50% inhibition of the cells.

The present disclosure is directed towards the treatment of various types of cancer or diseases by inhibiting the drug pump function of P-glycoprotein. P-glycoprotein is expressed with a high incidence in, but not limited to, colorectal, kidney, adrenocortical, breast, ovary, or hepatocellular cancers; sarcomas; and leukemia. The application of Schisandrin B to treat cancer has potential to treat other types of diseases in addition to cancer.

In consideration of the beneficial effect on the reversal of MDR cancer produced by the application of the compound Schisandrin B in anticancer medications, this compound may also be useful not only for therapeutic treatment after the onset of MDR, but also for prevention of MDR in patients about to undergo chemotherapy for the first time.

Anticancer medications prepared according to this disclosure may be formulated in various forms for administration, including, but not limited to, tablets, caplets, capsules, pills, suspensions, liquids and the like. As used herein, “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired.

Acceptable solid carriers may include one or more substances that may also act as flavoring agents, lubricants, solubilizers, suspending agents, fillers, glidants, compression aids, binders or tablet-disintegrating agents or an encapsulating material. In tablets, the active ingredient may be mixed with a carrier having the necessary compression properties in suitable proportions and compacted in the shape and size desired. Acceptable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Any suitable liquid carriers may be used in preparing solutions, suspensions, emulsions, syrups and elixirs according to the present disclosure. Schisandrin B, may be dissolved or suspended in any pharmaceutically acceptable liquid carrier such as water, one or more organic solvents, mixtures of both or pharmaceutically acceptable oils or fat. The liquid carrier may also include other suitable pharmaceutical additives such as solubilizers, emulsifiers, buffers, preservatives, viscosity regulators, stabilizers or osmoregulators. Exemplary liquid carriers for oral and parenteral administration include water (especially containing additives as above, e.g., cellulose derivative and sodium carboxymethyl cellulose solution), alcohols (e.g., monohydric alcohols and polyhydric alcohols) and their derivatives, and oils (e.g., fractionated coconut oil and arachis oil). For parenteral administration the pharmaceutically acceptable carrier may also be an oily ester such as ethyl oleate and isopropyl myristate. Sterile liquid carriers may also be used in sterile liquid form compositions for parenteral administration.

Any suitable liquid pharmaceutical compositions that are sterile solutions or suspensions may be utilized by, for example, intramuscular or subcutaneous injection. Sterile solutions can also be administered intravenously. Oral administration may be either in the form of a solid or liquid composition.

Schisandrin B according to the present disclosure may be administered using any amount and any route of administration effective for treating MDR cancer cells. The administration of a therapeutically effective amount is generally desirable. A therapeutically effective amount refers to a nontoxic but sufficient amount of the MDR reversal agent to provide the desired effect against the MDR cells. The exact amount will vary from subject to subject, depending on such factors as the species, age, general medical condition of the subject, the particular MDR reversal agent, its mode of administration and the like.

Although treatment using Schisandrin B according to the present disclosure described herein may be administered to any subject susceptible to the development of MDR, methods of treatment according to the present disclosure are intended particularly for the treatment of cancer in humans.

Schisandrin B compounds according to the present disclosure are extracted from the Chinese Schisandra chinensis (Turcz.) Baill and Schisandra sphenanthera Rehd. et Wils. plant according to various procedures well known to those of ordinary skill in the art.

In a first embodiment according to the present disclosure, a method of application of the compound Schisandrin B in reversing MDR cancer that includes preparing a medication comprising Schisandrin B is disclosed. It is contemplated by the present disclosure that the anticancer medication prepared with Schisandrin B can include at least one anticancer chemotherapeutic agent that can be combined with a pharmaceutically acceptable carrier as described herein. The anticancer chemotherapeutic agent may be selected from doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, Schisandrin B, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof. The anticancer medication prepared by the present disclosure can include Schisandrin B combined with other MDR reversal agents such as XR-9576, R-101933, and LY-335979 (Gottesman M M et al., Nat Rev/Cancer 2:48-58 (2001)).

In other embodiments, the present disclosure provides methods of application of the compound Schisandrin B for cancer therapy or cancer chemotherapy that includes preparing a medication comprising Schisandrin B. The embodiments set forth herein establish that the compound Schisandrin B is highly effective in inhibiting the drug-transport function of P-glycoprotein in MDR cancer cells. In particular, Schisandrin B inhibits the expression of P-glycoprotein in MDR cancer cells. By incorporating Schisandrin B into anticancer medications prepared by the methods of this disclosure, Schisandrin B binds with ABC transporter protein, P-glycoprotein, and effectively competes with anticancer agents in reversing MDR cancer. Schisandrin B further has the ability to induce apoptosis or death of cancer cells.

The following embodiments are intended to illustrate and not to limit the disclosure.

To illustrate a method for reversal of drug resistance by application of Schisandrin B, cell lines K562/adr, K562/vcr (X. Hu et al., Chemotherapy 41:296-305 (1995)), and KBv200 (X. H. Zhang et al., Yao Xue Xue Bao 29:246-251 (1994)) were selected for use because these particular cell lines are characteristic of overexpression of P-glycoprotein. Cells were cultured in RPMI-1640 medium containing 10% fetal calf serum. The test compound Schisandrin B was dissolved in dimethyl sulfoxide (DMSO) (10 mg/ml as stock). The logarithmic growing cells were seeded into a 96 well plate (20,000 cells/well). Specific anticancer drugs (set forth in Table 1) and Schisandrin B in combination were added into the wells containing the select cell lines. Controls for each anticancer drug type without Schisandrin B were also seeded into the 96 well plate. The cell culture was incubated for 48 hours in a humidified CO₂ incubator at 37° C. Cell viability was assessed by MTT assays as described in X. Hu et al., Chemotherapy 41:296-305 (1995).

As shown in Table 1, Schisandrin B has a relatively high potency in reversing drug resistance of MDR cancer cells K562/vcr. “Control” represents the IC₅₀ of anticancer drugs toward K562/vcr in the absence of Schisandrin B; “Schisandrin B” represents the IC₅₀ of anticancer drugs toward K562/vcr in the presence of Schisandrin B (10 μg/ml); “RF” (Reversal Folds) represents the IC₅₀ in the absence of Schisandrin B divided by the IC₅₀ in the presence of Schisandrin B. As indicated in Table 1, Schisandrin B is highly effective in reversing drug resistance of MDR cancer cell K562/vcr.

TABLE 1
IC₅₀ (μg/ml)

| | Control | Schisandrin B | RF |
|---|---|---|---|
| Doxorubicin | 4.89 ± 0.21 | 0.19 ± 0.01 | 25.7 |
| Daunorubicin | 0.45 ± 0.25 | 0.011 ± 0.0001 | 40.9 |
| Epirubicin | 2.56 ± 0.84 | 0.44 ± 0.14 | 5.81 |
| Mitoxantron | 0.79 ± 0.33 | 0.20 ± 0.06 | 4.0 |
| Vincristine | >20 | 1.34 ± 0.08 | >14.9 |
| Methotrexate | >10 | >10 | |
| Homoharringtonine | 0.55 ± 0.02 | 0.050 ± 0.01 | 11 |

In this embodiment, the IC₅₀ of anticancer drugs in the presence or absence of Schisandrin B toward MDR cells was determined. Verapamil, a potent MDR reversal agent (R. Krishna & L. D. Mayer., European J. Pharmacol. Sci. 11:265-283 (2000) and references therein), was used as a positive control.

The MDR cell lines of K562/adr, K562/vcr and KBV200 were selected because each respective cell line possesses the characteristics of P-glycoprotein overexpression. MTT assays were carried out in triplicate against human leukemia MDR cell line K562/adr and human epidermoid carcinoma MDR cell line KBV200. MTT assays as described previously (X. Hu, et al., Chemotherapy 41:296-305 (1995)) were used to determine the cytotoxicity of each anticancer drug in the presence or absence of Schisandrin B or verapmil. The treatment of the above cells lasted for 72 hours in a humidified CO₂ incubator at 37° C. The cell number in each sample was estimated by correlating to optical density at 595 nm. The median dose value was determined from plots of median effects and was equivalent to IC₅₀. Alternatively, flow cytometric assays were applied to count the cell numbers of each sample. The median dose value was determined from plots of median effects and was equivalent to IC₅₀.

As shown in Tables 2 and 3, the efficacy of Schisandrin B in reversing drug resistance is comparable to that of verapamil. Specifically, the potency of Schisandrin B in reversing vincristine resistance of KBV200 is about 5 folds more than that of verapamil.

TABLE 2
Reversal of drug resistance of K562/adr to
anticancer drugs by Schisandrin B

| | IC₅₀ (μg/ml) | | IC₅₀ (μg/ml) | | |
|---|---|---|---|---|---|
| Drug | Control | Schisandrin B | RF | verapamil | RF |
| Taxol | 0.3423 ± 0.1322 | 0.01913 ± 0.0208 | 17.89 | 0.0143 ± 0.0093 | 23.94 |
| Doxorubicin | 1.4175 ± 0.2463 | 0.0691 ± 0.0137 | 20.57 | 0.1324 ± 0.0086 | 10.76 |
| Mitoxantrone | 0.1052 ± 0.0356 | <0.01 | >10.52 | <0.01 | >10.52 |
| Daunorubicin | 0.5046 ± 0.0728 | 0.0977 ± 0.063 | 5.16 | 0.0791 ± 0.0313 | 6.38 |
| Vincristine | 0.9162 ± 0.238 | 0.1486 ± 0.0554 | 6.2 | 0.0872 ± 0.0236 | 10.51 |
| Epirubicin | 1.3196 ± 0.3218 | 0.3033 ± 0.0812 | 4.35 | 0.2218 ± 0.0909 | 5.95 |
| Homoharringtonine | 0.4283 ± 0.1079 | 0.0964 ± 0.0312 | 4.44 | 0.0701 ± 0.0042 | 6.1 |
| Hydroxycamptothecine | 0.3688 ± 0.0875 | 0.0417 ± 0.062 | 8.84 | 0.0432 ± 0.0205 | 8.53 |

Table 2 sets forth the results of K562/adr cells treated with anticancer drugs in the absence or presence of Schisandrin B or verapamil. Cells were treated with specific anticancer drugs as set forth therein. “Ctrl” represents control cells treated with anticancer drugs in the absence of Schisandrin B or verapamil. “SchB” represents cells treated with anticancer drugs in the presence of Schisandrin B (10 μg/ml). “Ver” represents cells treated with anticancer drugs in the presence of verapamil (6 μg/ml). “RF” represents Reversal Folds of drug resistance, IC₅₀ in the absence of Schisandrin B or Verapamil divided by IC₅₀ in the presence of Schisandrin B or verapamil.

TABLE 3
Reversal of drug resistance of MDR cell KBV200 by Schisandrin B

| | IC₅₀ (μg/ml) | | IC₅₀ (μg/ml) | | |
|---|---|---|---|---|---|
| Drug | Control | Schisandrin B | RF | verapamil | RF |
| Doxorubicin | 0.3272 ± 0.2131 | 0.0691 ± 0.0137 | 4.74 | 0.0324 ± 0.0086 | 10.11 |
| Mitoxantron | 0.0906 ± 0.0456 | <0.01 | >9.06 | <0.01 | >9.06 |
| Daunorubicin | 0.3046 ± 0.0728 | 0.0913 ± 0.003 | 3.34 | 0.0555 ± 0.0175 | 5.49 |
| Vincristine | >20 | 0.1572 ± 0.0946 | 127.2 | 0.9872 ± 0.01555 | 20.26 |
| Epirubicin | 0.9876 ± 0.1524 | 0.1033 ± 0.0545 | 9.56 | 0.1226 ± 0.082 | 8.06 |
| Homoharringtonine | 0.5257 ± 0.1987 | 0.1064 ± 0.0812 | 4.94 | 0.1108 ± 0.0076 | 4.74 |
| Hydroxycamptothecine | 0.1618 ± 0.1501 | 0.0527 ± 0.0613 | 3.07 | 0.0468 ± 0.0254 | 3.48 |
| Taxol | 0.0890 ± 0.0079 | 0.0316 ± 0.0019 | 2.82 | 0.0329 ± 0.0079 | 2.71 |

Table 3 sets forth the results of MDR KBV200 cells treated with anticancer drugs in the absence or presence of Schisandrin B or verapamil. Cells were treated with specific anticancer drugs as set forth therein. “Ctrl” represents control cells treated with anticancer drugs in the absence of Schisandrin B or verapamil. “SchB” represents cells treated with anticancer drugs in the presence of Schisandrin B (10 μg/ml). “Ver” represents cells treated with anticancer drugs in the presence of verapamil (6 μg/ml). “RF” represents Reversal Folds of drug resistance, IC₅₀ in the absence of Schisandrin B or verapamil/IC₅₀ in the presence of Schisandrin B or verapamil.

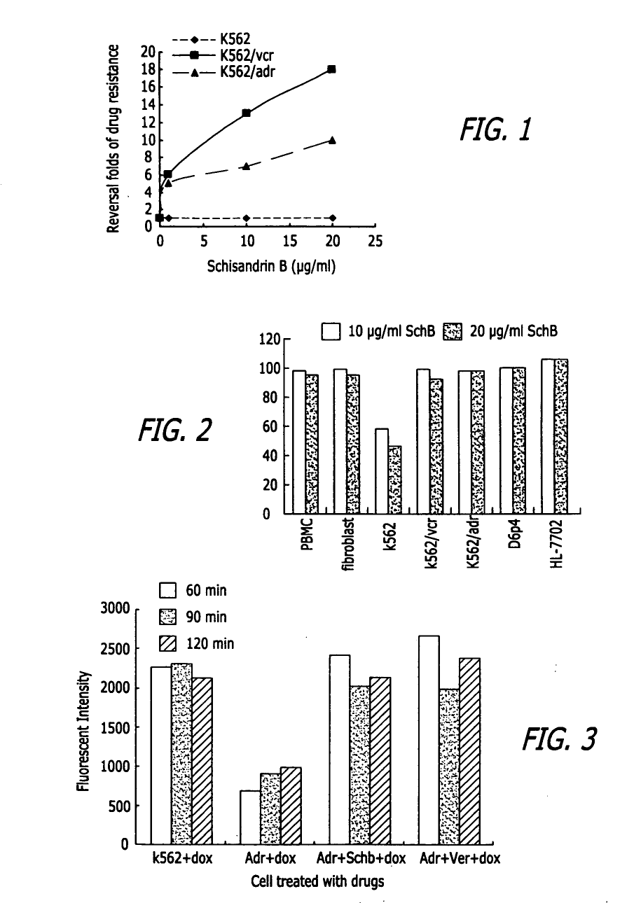

According to the present disclosure, Schisandrin B greatly increased the sensitivity of MDR cancer cell K562/adr and K562/vcr to doxorubicin. For instance, in the presence of Schisandrin B at concentrations of 1, 10, 20 μg/ml, the sensitivity of K562/vcr to doxorubicin increased to about 5, 12, and 18 folds, respectively, indicating Schisandrin B was able to effectively reverse drug resistance mediated by P-glycoprotein (FIG. 1).

To illustrate the cytotoxicity of Schisandrin B according to the present disclosure, cell lines selected for use include cancer cell lines K562, K562/Adr, K562/vcr; primary cultured cells, including human peripheral blood mononuclear cells (PBMC) and human fibroblast cells; noncancer cell lines, including human bone marrow stromal cell D6p4 and human liver cell HL-7702. Cells were cultured in RPMI-1640 medium containing 10% fetal calf serum. The logarithmic growing cells were seeded into a 96 well plate (20,000 cells/well). Cells were incubated for 48 hours in a humidified CO₂ incubator at 37° C. in the presence of 0, 10, and 20 μg/ml Schisandrin B. Cell viability was assessed by MTT assays as described in X. Hu, et al., Chemotherapy 41:296-305 (1995).

As illustrated in FIG. 2, at the effective concentration of Schisandrin B to reverse drug resistance, Schisandrin B does not demonstrate cytotoxicity toward normal human cells and MDR cells but does demonstrate cytotoxicity toward K562 cells.

Referring now to FIG. 3, intracellular accumulation of doxorubicin in MDR cancer cell K562/adr according to the present disclosure is illustrated.

The cell line selected was MDR cancer cell K562/adr and its parental drug sensitive cell K562. Cells were cultured in RPMI-1640 medium containing 10% fetal calf serum in cell-culture flasks. Cells were incubated with the anticancer drug doxorubicin (5 μg/ml) in the presence or absence of Schisandrin B (10 μg/ml) or verapamil (6 μg/ml) for 60, 90 and 120 minutes at 37° C. in a humidified CO₂ incubator. Following the respective incubation period for each cell line, the cells were collected and the intracellular doxorubicin concentration was determined by flow cytometry using FACS Calibur equipped with software Cellquest 3.1f.

According to the present disclosure, the drug sensitive cells accumulated significantly more intracellular doxorubicin than MDR cell K562/adr in the absence of Schisandrin B or verapamil. In the presence of Schisandrin B, the pump function of P-glycoprotein was inhibited, resulting in significant intracellular drug accumulation in K562/adr cells comparable to K562 cells. The potency of Schisandrin B is comparable to verapamil in restoring drug accumulation in MDR cell K562/adr. FIG. 3 illustrates that Schisandrin B restored intracellular doxorubicin accumulation in K562/adr, and its potency is comparable with verapamil. Specifically, in FIG. 3, “K562+dox” represents K562 cell and 5 μg/ml doxorubicin; “Adr+dox” represents K562/adr and 5 μg/ml doxorubicin; “Adr+SchB+Dox” represents K562/adr and 5 μg/ml doxorubicin and 10 μg/ml Schisandrin B; “Adr+Ver+Dox” represents K562/adr and 5 μg/ml doxorubicin and 10 μg/ml verapamil.

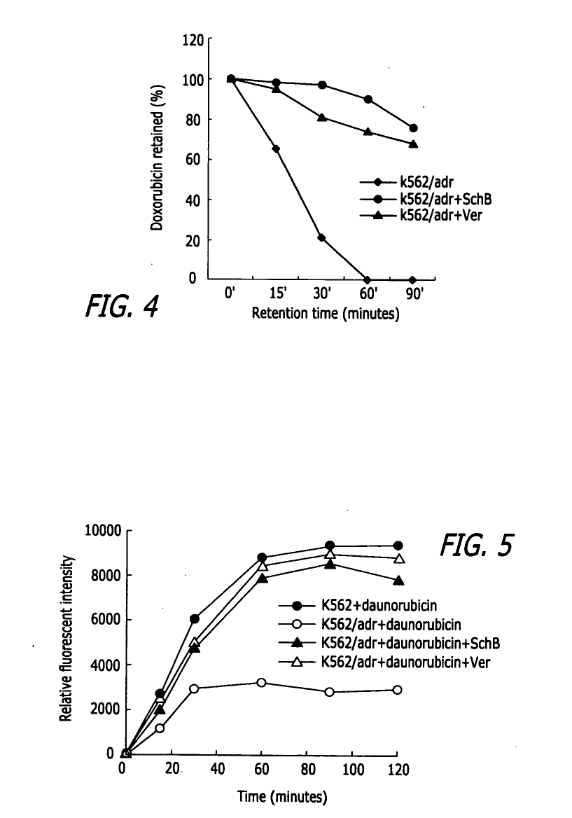

Referring now to FIG. 4, efflux of doxorubicin in MDR cell K562/adr is illustrated.

Drug retention within cells was assayed by loading K562/adr or K562 cells with doxorubicin (2 μg/ml) for 60 minutes at 37° C. in a humidified CO₂ incubator. Cells were collected and suspended into RPMI-1640 complete medium in the presence or absence of Schisandrin B (10 μg/ml) or verapamil (6 μg/ml) and then incubated at 37° C. in a humidified CO₂ incubator. At incremental times of 15, 30, 60 and 90 minutes, respectively, cells were collected and the doxorubicin concentration within the cells was measured by reversed phase High Performance Liquid Chromatography (HPLC) as described in X. Hu et al., Acta Pharmaceutica Sinica 29:246-251 (1994).

In the absence of Schisandrin B or verapamil, K562/adr effluxed doxorubicin very quickly out of cells. At 60 minutes of incubation, K562/adr almost completely expelled doxorubicin from cells. In the presence of Schisandrin B or verapamil, however, intracellular doxorubicin was retained. At 90 minutes of incubation, about 80% of intracellular doxorubicin was retained in K562/adr cells. FIG. 4 illustrates that Schisandrin B inhibited intracellular doxorubicin efflux of K562/adr, and its potency is comparable with verapamil.

The following embodiment illustrates that Schisandrin B inhibits P-glycoprotein mediated drug efflux in MDR cancer according to the present disclosure.

P-glycoprotein functions as a drug pump that unilaterally pumps the anticancer drugs out of MDR cancer cells. Inhibition of P-glycoprotein results in an increase of the intracellular drug concentration within cancer cells. The inhibition of P-glycoprotein is assessed by analyzing the anticancer drug concentration within the test cells in the presence or absence of Schisandrin B. Verapamil, a potent P-glycoprotein inhibitor, was used as a positive control.

MDR cancer cell K562/adr and its drug sensitive parental cell K562 were separately incubated in RPMI-1640 complete medium containing 2 μg/ml daunorubicin in the presence or absence of Schisandrin B (10 μg/ml) or verapamil (6 μg/ml) at 37° C. Cells were collected at intervals of 15, 30, 60, 90, 120, and 160 minutes after incubation. Cells were washed twice with ice-cold phosphate buffered saline and the daunorubicin concentration within cells was measured by flow cytometry at excitation wavelength of 488 nm and emission wavelength of 533 nm using a FACS Calibur equipped with software Cellquest 3.1f (Becton-Dickinson, Holbrook, N.J.).

Alternatively, MDR cancer cell KBV200 and its drug-sensitive parental cell KB were separately incubated in RPMI-1640 complete medium containing 2 μg/ml Rhodamine-123 (Rh-123) in the presence or absence of Schisandrin B (10 μg/ml) or verapmil (6 μg/ml) at 37° C. Cells were collected at intervals of 15, 30, 60, 90, and 120 minutes after incubation. Cells were washed twice with ice-cold phosphate buffered saline and the Rh-123 concentration within cells was measured by flow cytometry at excitation wavelength of 488 nm and emission wavelength of 533 nm using FACS Calibur equipped with software Cellquest 3.1f (Becton-Dickinson, Holbrook, N.J.). At least 50,000 cells were analyzed for each sample.

FIG. 5 illustrates that Schisandrin B restored intracellular daunorubicin accumulation in K562/adr, and its potency is comparable with verapamil.

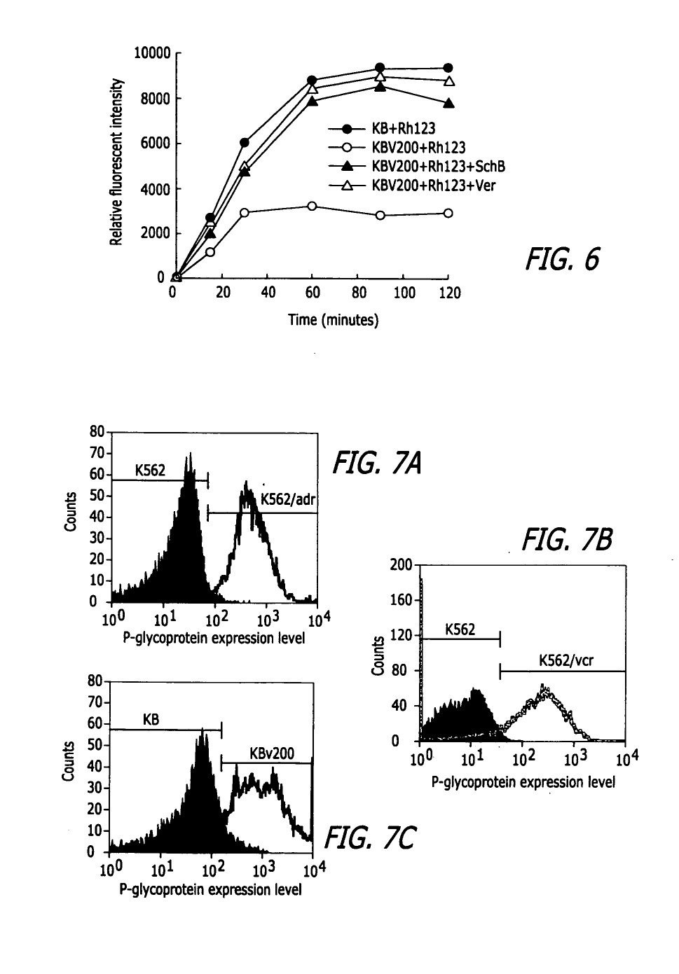

FIG. 6 illustrates that Schisandrin B restored intracellular Rh-123 accumulation in KBV200, and its potency is comparable with verapamil.

This embodiment according to the present disclosure demonstrates that Schisandrin B is able to completely inhibit the drug-efflux function of P-glycoprotein, and fully restore the intracellular drug accumulation of MDR cancer cells.

Referring now to FIGS. 7A, 7B, and 7C, detection of P-glycoprotein expression in MDR cell lines according to the present disclosure is illustrated.

Drug sensitive cell line, K562, and MDR cell lines, K562/adr, K562/vcr and KBV200, with the characteristics of P-glycoprotein overexpression, were used. Cells were suspended into phosphate buffered saline and adjusted to a cell density of 1×10⁶/ml. The cells were labeled with R-phycoerythrin conjugated mouse monoclonal antibody against human P-glycoprotein (Becton-Dickinson, Holbrook, N.J.) according to the manufacturer's instructions and subjected to flow cytometry analysis. The nonspecific labeling was corrected by the corresponding subtype control immunoglobulins. Flow cytometry was conducted using FACS Calibur equipped with software Cellquest 3.1f.

FIG. 7A illustrates that 98.19% K562/adr cells express P-glycoprotein while the average fluorescent intensity per cell is 611.43, and that 0.93% of K562 cells express P-glycoprotein while the average fluorescent intensity per cell is 25.48. FIG. 7B illustrates that 96.23% K562/vcr cells express P-glycoprotein while the average fluorescent intensity per cell is 531.23. FIG. 7C illustrates that 95.94% KBV200 cells express P-glycoprotein while the average fluorescent intensity per cell is 1480.68, and that 2.3% KB cells express P-glycoprotein while the average fluorescent intensity per cell is 53.64. The results confirmed that K562/adr, K562/vcr, and KBV200 cell lines are typical MDR cancer cell lines with the characteristics of P-glycoprotein overexpression.

To illustrate the effects of Schisandrin B on the intracellular distribution in MDR cancer cells, MDR cell KBV200 and its parental drug sensitive cell line KB were selected for use. The anticancer drug, daunorubicin, and fluorescent indicator, Rhodamin Rh-123, are fluorescent at an excitation wavelength of 488 nm and emission wavelength of 533 nm. Because of its fluorescent properties, the cellular distribution of daunorubicin and Rh-123 within cells may be directly observed under fluorescent microscope. Daunorubicin or Rh-123 was added into a KB (1×10⁶ cells/ml) or KBV200 (1×10⁶ cells/ml) cell suspension at a final concentration 2 μg/ml in the presence or absence of schisandrin B or verapamil. The respective cultures were incubated at 37° C. for 1 hour, washed with ice-cold PBS, and the cells were observed under fluorescent microscope.

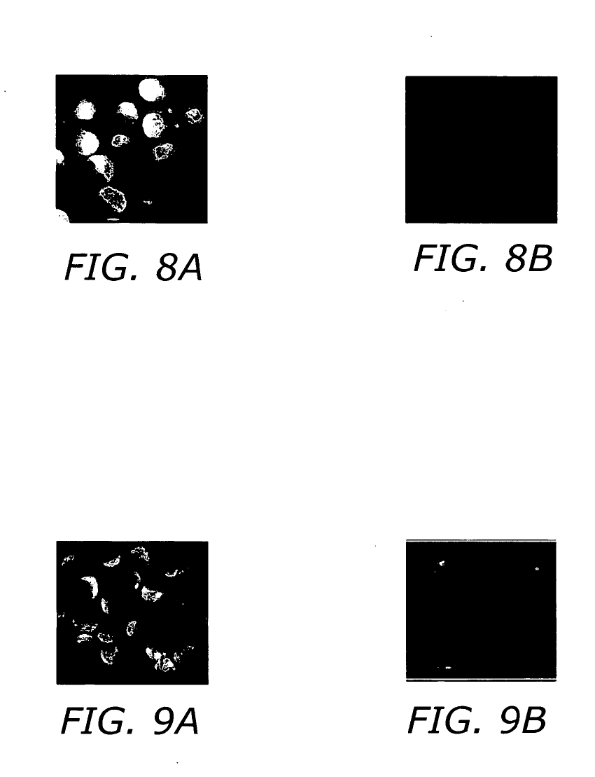

Under fluorescent microscope, it was observed that daunorubicin was primarily distributed in the nuclei of KB cells, while it was not detected in the nuclei of KBV200, indicating that daunorubicin was not able to reach its target in MDR cancer cells (FIGS. 8A and 8B). FIGS. 8A and 8B illustrate the intracellular distribution of daunorubicin in KB and KBV200 cells, respectively. Similar results were observed when Rh-123 was used as a fluorescent indicator (FIGS. 9A and 9B). FIGS. 9A and 9B illustrate the intracellular distribution of Rh-123 in KB and KBV200 cells, respectively.

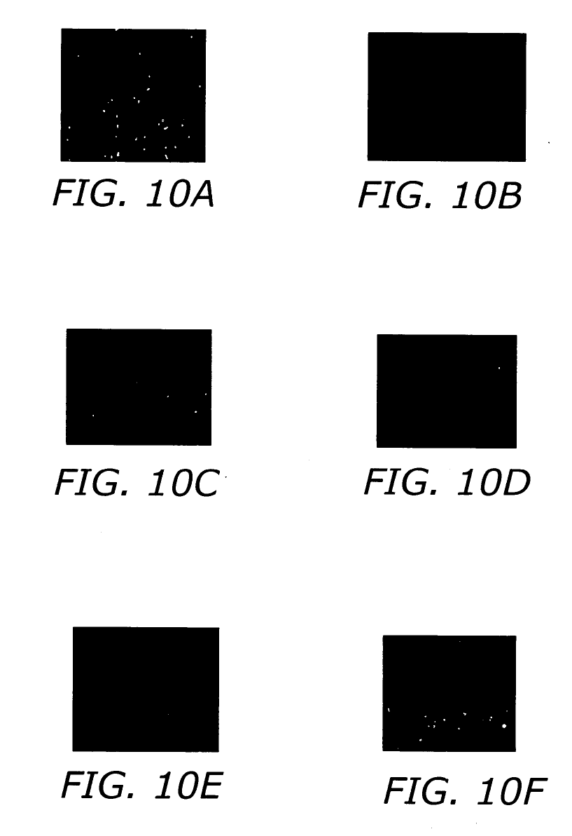

In the presence of Schisandrin B, the intracellular distribution of daunorubicin and Rh-123 was partially restored and daunorubicin was detected in nuclei of MDR cancer cells (FIGS. 10A-F and 11A-D). In particular, FIGS. 10A-F illustrate via fluorescent microscope the effects of Schisandrin B or verapamil on the intracellular distribution of daunorubicin in KBV200 cells.

FIGS. 10A and 10B illustrate the daunorubicin accumulation in the presence of verapamil (10 μg/ml) at magnification of ×100 and ×200, respectively. FIGS. 10C and 10D illustrate the daunorubicin accumulation in the presence Schisandrin B (5 μg/ml) at magnifications of ×100 and ×200, respectively. FIGS. 10E and 10F illustrate the daunorubicin accumulation in the presence of Schisandrin B (10 μg/ml) at magnifications of ×100 and ×200, respectively.

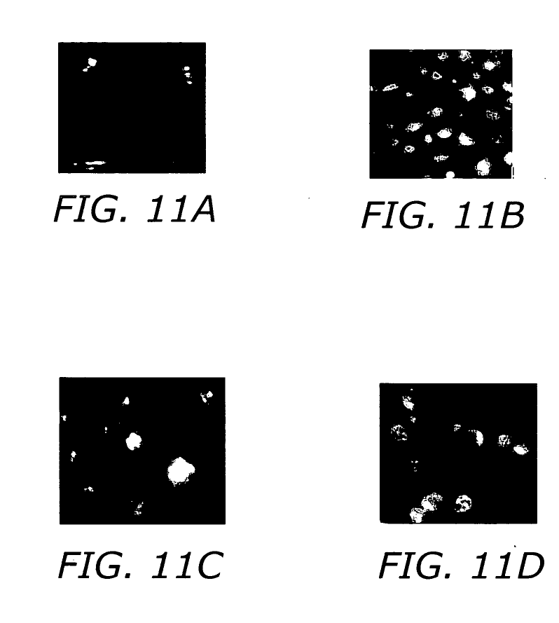

FIGS. 11A-D illustrate via fluorescent microscope the effects of Schisandrin B or verapamil on the intracellular distribution of Rh-123 in KBV200 cells. FIG. 11A illustrates the Rh-123 accumulation in KBV200 cells in the absence of Schisandrin B or verapamil. FIG. 11B illustrates the Rh-123 accumulation in KBV200 cells in the presence of verapamil (10 μg/ml). FIG. 11C illustrates the Rh-123 accumulation in KBV200 in the presence of Schisandrin B (5 μg/ml). FIG. 11D illustrates the Rh-123 accumulation in KBV200 cells in the presence of Schisandrin B (10 μg/ml).

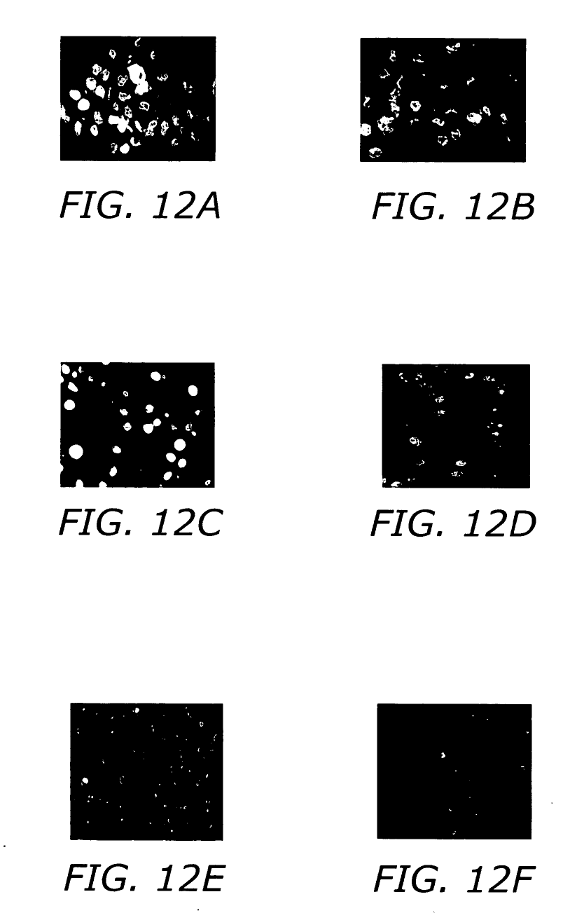

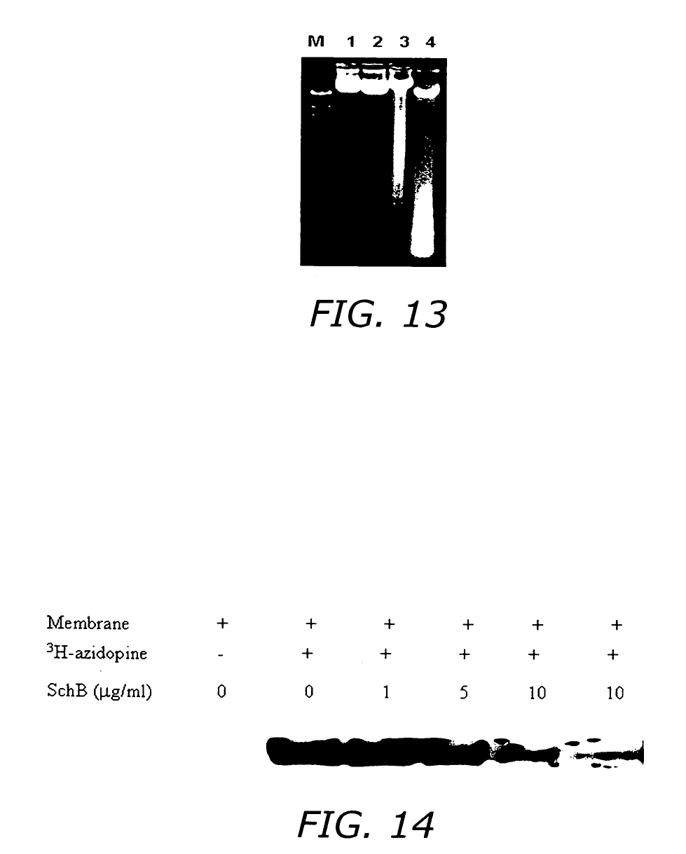

Referring now to FIGS. 12 and 13, Schisandrin B enhancing the apoptosis of MDR cancer cells induced by doxorubicin according to the present disclosure is illustrated.

MDR cancer cells, KBV200, were incubated in RPMI-1640 complete medium containing anticancer drug doxorubicin at concentrations of 1 μg/ml and 5 μg/ml in the presence or absence of Schisandrin B (10 μg/ml) for 24 hours in a humidified CO₂ incubator at 37° C. Cells were collected and stained with fluorescent dyes Hoechst 33342 and propidium iodide. Hoechst 33342 stains the nuclei of cells with the color blue, and propidium iodide exclusively stains dead cells with the color of red. Accordingly, while Hoechst 33342 only stained the living cells blue, the dead cells were stained by both dyes with the colors of yellow, orange or red. The percentage of dead cells was counted under fluorescent microscope.

As shown in FIG. 12 and Table 4, doxorubicin at 1 μg/ml failed to cause death of MDR cell KBV200. Doxorubicin at 5 μg/ml caused a small percentage of death of MDR cell KBV200. However, in the presence of Schisandrin B, doxorubicin at 1 μg/ml caused about 50% death of the MDR cell KBV200.

FIG. 12 illustrates the apoptosis of KBV200 cells treated with doxorubicin in the presence or absence of Schisandrin B. FIG. 12A shows untreated KBV200 cells; FIG. 12B shows KBV200 cells treated with doxorubicin (1 μg/ml); FIG. 12C shows KBV200 cells treated with doxorubicin (5 μg/ml); FIG. 12D shows KBV200 cells treated with doxorubicin (1 μg/ml) and Schisandrin B (10 μg/ml); FIG. 12E shows KBV200 cells treated with doxorubicin (5 μg/ml) and Schisandrin B (10 μg/ml); and FIG. 12F shows KB cells treated with doxorubicin (1 μg/ml).

TABLE 4
The effects of Schisandrin B on the apoptosis of
KB and KBV200 induced by doxorubicin
% apoptosis

| Cell | Control | A1 | A5 | A1 + S10 | A5 + S10 |
|---|---|---|---|---|---|
| KBV200 | 1.2% | 1.7% | 7.6% | 46.1% | >90% |
| KB | 0.8% | >90% | >90% | | |

With reference to Table 4, “Ctrl” represents cells not treated (control); “A1” represents cells treated with doxorubicin (1 μg/ml); “A5” represents cells treated with doxorubicin (5 μg/ml); “A1+S10” represents cells treated with doxorubicin (1 μg/ml) and Schisandrin B (10 μg/ml); and “A5+S10” represents cells treated with doxorubicin (5 μg/ml) and Schisandrin B (10 μg/ml).

These results establish that Schisandrin B is able to enhance the activities of select anticancer drugs against MDR cancer cells, or, Schisandrin B is able to synergistically corporate with select anticancer drugs in killing MDR cancer cells.

The test cells were also analyzed by flow cytometry after being subjected to propidium iodide staining. Similar results were obtained (Table 5).

TABLE 5
The effects of Schisandrin B on the apoptosis (hypodiploid) of
KBV200 induced by doxorubicin
Cell cycle distribution (%)

| Treatment | G0/G1 | S | G2/M | hypodiploid % |
|---|---|---|---|---|
| A1 | 44.76 | 45.09 | 10.14 | 1.21 |
| A5 | 48.03 | 42.37 | 9.60 | 7.51 |
| A1□S10 | 54.04 | 40.30 | 5.66 | 24.10 |
| A5□S10 | 58.94 | 39.36 | 1.70 | 70.31 |

With reference to Table 5, “A1” represents cells treated with doxorubicin (1 μg/ml); “A5” represents cells treated with doxorubicin (5 μg/ml); “A1+S10” represents cells treated with doxorubicin (1 μg/ml) and Schisandrin B (10 μg/ml); and “A5+S10” represents cells treated with doxorubicin (5 μg/ml) and Schisandrin B (10 μg/ml).

The DNA of above cells was also extracted and subjected to agarose electrophoresis. The results indicate that, while KBV200 cells treated with doxorubicin combined with Schisandrin B demonstrated DNA fragmentation (FIG. 13), a hallmark for apoptosis, they did not show the similar DNA fragmentation in the absence of Schisandrin B.

FIG. 13 illustrates the effects of Schisandrin B on enhancing the activities of doxorubicin in the induction of apoptosis of KBV200 cells. With further reference to FIG. 13, “M” represents the molecular marker; Lane “1” represents control cells (untreated); Lane “2” represents cells treated with doxorubicin (5 μg/ml); Lane “3” represents cells treated with doxorubicin (1 μg/ml) and Schisandrin B (10 μg/ml); and Lane “4” represents cells treated with doxorubicin (5 μg/ml) and Schisandrin B (10 μg/ml).

To illustrate that Schisandrin B physically interacts with P-glycoprotein according to the present disclosure, [³H]Azidopine Photoaffinity Labeling of P-glycoportein in the presence or absence of Schisandrin B was used. [³H]Azidopine is a chemical having unique properties that enable it to interact with P-glycoprotein. If Schisandrin B is also able to interact with P-glycoprotein, the binding of P-glycoprotein with [³H]Azidopine would be reduced in the presence of Schisandrin B.

The cytoplasmic membranes (enriched with P-glycoprotein) of MDR cell KBV200 were prepared according to the methods as described in May GL, et al., Int. J. Cancer 42:728-733(1988) and Hyafil F, et al., Cancer Res. 53:4595-4602(1993). Membranes were incubated with Schisandrin B for 40 minutes in the dark, followed by 1 hour incubation with 1.0 μM [H]azidopine. After UV irradiation for 2 minutes, the photolabeled membranes were subjected to SDS-PAGE on a 7.5% gel, followed by fluorography.

As illustrated in FIG. 14, [³H]Azidopine binding with P-glycoprotein was inhibited in the presence of Schisandrin B. [³H]azidopine binding with P-glycoprotein decreased with the increasing concentration of Schisandrin B, demonstrating dose and effect relationship (FIG. 14).

The results show that Schisandrin B is able to physically interact with P-glycoprotein.

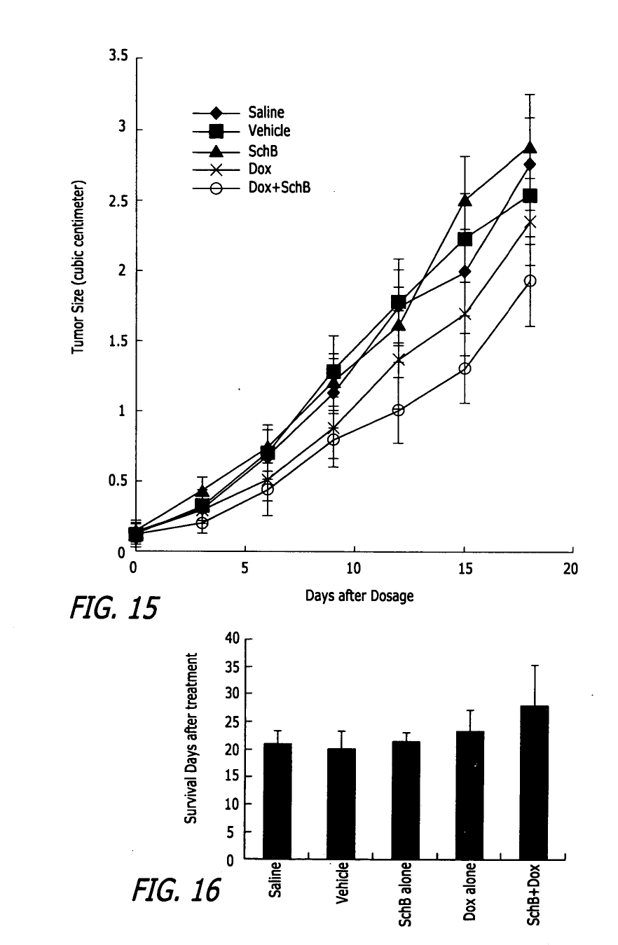

Referring now to FIG. 15, in vivo efficacy of Schisandrin B in reversing drug resistance of MDR cancer according to the present disclosure is illustrated.

Thirty nine Balb/c nu/nu female mice were inoculated subcutaneously with KBV200 cells (5×10⁶ KBV200 cells/each mouse) on day 0. On day 10, the mice were randomly divided into 5 groups as follows: saline, vehicle, Schisandrin B (50 mg/kg) alone, doxorubicin alone, and doxorubicin combined with Schisandrin B. The dose schedule consisted of the administration of the drugs on days 11, 14, 18, 21 and 25. Thirty minutes before injection of doxorubicin (5 mg/kg) via tail vein, mice of the 5 groups were administered via gastric intubation with 100 μl saline, vehicle (PEG400:dextrose 7:3 by volume), Schisandrin B (2 mg in vehicle), saline, and Schisandrin B (2 mg in vehicle), respectively. Mean body weights were recorded daily. All mice were observed once per day (or more) for mortality and signs of ill health (i.e., weight loss, change in appetite, or behavioral changes). Therapeutic comparisons were made between control and treatment groups by determining the tumor size. Tumor size was monitored approximately every other day by caliper measurements and calculated according to the formula [Tumor weight=(length×width²)÷2]. The median survival times (MST) and the percent increase in life span (% ILS) were calculated as: % ILS=[(MSTₜᵣₑₐₜₘₑₙₜ/MST_{control})−1]×100. Statistical significance between the various groups was determined by log rank analysis using a significance criterion of P<0.05 (M. J. Newman, et al., Cancer Res. 60:2964-2972 (2000)).

With further reference to FIG. 15, after mice were inoculated with human MDR KBv200 cancer cells, mice were treated with doxorubicin alone or doxorubicin combined with Schisandrin B. Whereas the tumor sizes in mice treated with doxorubicin alone were not significantly different from those of control groups (i.e., saline, vehicle, or Schisandrin B alone), the tumor sizes in mice treated with doxorubicin combined with Schisandrin B were significantly smaller than control groups, indicating Schisandrin B was able to effectively reverse drug resistance of MDR cancer in the in vivo model.

As shown in Table 6 and FIG. 16, the median survival times of mice bearing MDR cancer cell KBV200 treated with doxorubicin alone was not significantly prolonged as compared with the control mice. However, the median survival times of mice bearing MDR cancer cell KBV200 treated with doxorubicin combined with Schisandrin B was significantly prolonged as compared with the control mice.

TABLE 6
Effect of Schisandrin B on MST and T/C % in Balb/c
nude mice bearing KBV200-implanted tumor

| Group | No. of Animals | MST (days) | T/C (%) |
|---|---|---|---|
| Saline | 7 | 20.8 | 100 |
| Vehicle | 7 | 19.9 | 95.7 |
| SchB alone | 7 | 21.2 | 101.9 |
| Dox alone | 8 | 23.1 | 111.1 |
| Dox + SchB | 10 | 27.9 | 134.1* |

Table 6 summarizes the effect of Schisandrin B on MST and T/C % in Balb/c nude mice bearing KBV200-implanted tumor. “MST” represents median survival time. The survival rate (T/C %) was calculated by following equation: T/C(%)=[Average survival period in the test group/average survival period in the control group]×100. *P<0.05 compared to saline, vehicle, or Schisandrin B alone. These results prove that Schisandrin B demonstrated satisfactory efficacies in reversing P-glycoprotein-mediated drug resistance of MDR cancer in the in vivo model.

In summary, Schisandrin B is able to effectively reverse cancer multidrug resistance in both the in vitro and in vivo models and its potency is comparable with verapamil. While verapamil is of dose-limiting side effects, Schisandrin B is of high safety. In view of the aforementioned embodiments, Schisandrin B is of future clinical application as an effective MDR reversal agent.

Having now described the disclosure in accordance with the requirements of the patent statutes, those skilled in this art will understand how to make changes and modifications in the present disclosure to meet their specific requirements or conditions. Such changes and modifications may be made without departing from the scope and spirit of the disclosure as set forth in the following claims.

1. A method of application of the compound Schisandrin B in reversing multidrug resistant cancer comprising:
preparing a medication comprising Schisandrin B.

2. The method of claim 1, wherein preparing the medication further comprises:
incorporating at least one anticancer chemotherapeutic agent and a pharmaceutically acceptable carrier.

3. The method of claim 2, wherein the at least one anticancer chemotherapeutic agent is selected from the group consisting of:
doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, Schisandrin B, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

4. The method of claim 1, wherein preparing the medication comprises:
formulating the medication for administration in the form of a capsule, caplet, tablet, pill, suspension or liquid.

5. The method of claim 1, wherein preparing the medication comprises:
incorporating at least one multidrug resistant reversal agent.

6. A method of application of the compound Schisandrin B for cancer therapy comprising:
preparing a medication comprising Schisandrin B.

7. The method of claim 6, wherein preparing the medication comprising Schisandrin B further comprises:
inhibiting the drug-transport function of P-glycoprotein in multidrug resistant cancer cells through Schisandrin B.

8. The method of claim 6, wherein preparing the medication comprising Schisandrin B further comprises:
binding Schisandrin B with P-glycoprotein by competing with anticancer agents.

9. The method of claim 8, wherein preparing the medication comprising Schisandrin B further comprises:
inducing apoptosis of cancer cells through Schisandrin B.

10. A method of application of the compound Schisandrin B for cancer chemotherapy comprising:
preparing a medication comprising Schisandrin B.
